(19) United States
(12) Patent Application Publication
(10) Pub. No.: US 2013/0344502 A1
(43) Pub. Date: Dec. 26, 2013

(54) VIBRATING MICROPLATE BIOSENSING FOR CHARACTERISING PROPERTIES OF BEHAVIOUR BIOLOGICAL CELLS

(71) Applicants: Xianghong Ma, Sambourne (GB); Helen R. Griffiths, Dorridge (GB)

(72) Inventors: Xianghong Ma, Sambourne (GB); Helen R. Griffiths, Dorridge (GB)

(73) Assignee: ASTON UNIVERSITY, Birmingham (GB)

(21) Appl. No.: 14/004,744

(22) PCT Filed: Mar. 15, 2012
(86) PCT No.: PCT/GB12/50571

§ 371 Date: Sep. 12, 2013

(30) Foreign Application Priority Data
Mar. 15, 2011 (GB) 1104462.5

(51) Int. Cl. G01N 33/543

(52) U.S. Cl. CPC G01N 33/54306
USPC 435/7.2; 436/501; 435/287.2; 422/69

(57) ABSTRACT

There is described a system for testing a sample. The system comprises a microplate, at least one actuator, a plurality of mutually spaced sensors, and a processor. The microplate has a test portion including an interactive substance. The interactive substance is inherently interactive with a specified test substance. The microplate is arranged such that at least the test portion of the microplate may be brought into contact with the sample. The at least one actuator is operable to vibrate the microplate. The plurality of mutually spaced sensors are coupled to the microplate. Each sensor is operable to provide a respective sensory data time series during vibration of the microplate. The microplate and the sensors are arranged such that the provided sensory data time series are not independent from one another. The processor is operable to receive the sensory data time series from the sensors and to process the received sensory data time series so as to provide information about the test substance in the sample based on the sensed interaction between the test substance and the interactive substance on the test portion of the microplate. A corresponding method of testing a sample is also described.

### FIELD OF THE INVENTION

The present invention relates to a system and method for testing a sample. For example, the system and method of the present invention may be used to test a biological sample (e.g. blood, urine, saliva, etc.) for the presence of a specific biological cell (e.g. a specific antigen).

### BACKGROUND OF THE INVENTION

In the UK, there are about 5,200 National Health Service (NHS) General Practitioner (GP) practices and about 900 private GP practices. Around the world there are many more. All GP clinics have nurses rooms used for taking samples, but almost all biological samples are then sent away to laboratories for medical testing. For example, in the UK, GP clinics routinely send tens of thousands of biological samples for testing (e.g. viruses, bacteria, proteins, sugar, etc. in blood, urine, semen, etc.) to third party laboratories each year. This practice is expensive and is also time consuming—it may take up to 2 weeks—and slows diagnosis and provision of appropriate treatment. In addition, existing testing technologies require calibration and need highly trained personnel. Therefore, there is a need for quicker in-house medical testing systems and methodologies to reduce costs and to improve patient service.

### SUMMARY OF THE INVENTION

According to a first aspect of the present invention, there is provided a system for testing a sample. The system comprises a microplate, at least one actuator, a plurality of mutually spaced sensors, and a processor. The microplate has a test portion including an interactive substance that is inherently interactive with a specified test substance. The microplate is arranged such that at least the test portion of the microplate may be brought into contact with the sample. The at least one actuator is operable to vibrate the microplate. The plurality of mutually spaced sensors are coupled to the microplate. Each sensor is operable to provide a respective sensory data time series during vibration of the microplate. The microplate and the sensors are arranged such that the provided sensory data time series are not independent from one another. The processor is operable to receive the sensory data time series from the sensors and to process the received sensory data time series so as to provide information about the test substance in the sample based on the sensed interaction between the test substance and the interactive substance on the test portion of the microplate.

The claimed system may be used in a clinical testing environment, especially GP clinics. For example, there is an imperative to reduce use of antibiotics, and this device could rapidly distinguish between virus and bacteria, so reducing antibiotic prescriptions. The claimed system may also be used in a wide range of other environments. Other potential fields of use include water testing, food testing, agricultural/veterinary testing, defence, etc.

The microplate may be a silicon microplate or a polymer microplate. The test portion of the microplate may be coated with the interactive substance. The microplate boundary conditions may be selected from clamped, cantilever, free and point-supported. The sensors may be selected from piezoresistive gauge sensors, optical sensors, strain sensors and acceleration sensors. The at least one actuator may comprise a piezoelectric transducer and/or a sonic actuator. The at least one actuator may be operable to vibrate the microplate periodically or randomly. The processor may be operable to analyse the sensory data time series in one or more of the time domain, the frequency domain and the wavelet domain. For example, the processor may be operable to analyse frequency response functions (FRFs). The processor may be operable to use one or more of a neural network and Karhunen-Loeve decomposition in the processing of the sensory data time series.

The microplate may be a disposable microplate for use in analysing a single sample, the disposable microplate being replaceable with another disposable microplate after use. Alternatively, the microplate may be re-usable for analysing a plurality of samples.

Advantageously, the system comprises a handheld testing device including the microplate, the at least one actuator, and the plurality of mutually spaced sensors. In one embodiment, the handheld testing device further includes the processor. In another embodiment, the processor is separate from the handheld testing device, and the handheld testing device is arranged to be coupled to the processor. In yet another embodiment, the processor is separate from the handheld testing device, and the handheld testing device is arranged to write the sensory data time series on a removable memory component that is readable by the processor.

The system may further comprise an output device, such as a display, speaker or printer, operable to output to a user any test results received from the processor. The output device may form part of the handheld testing device or may be separate from the handheld testing device. Preferably, the output device forms part of the handheld testing device when the processor also forms part of the handheld testing device.

The interaction between the interactive substance and the test substance may take many forms. Importantly, the interaction leads to some form of coupling between the interactive substance and the test substance such that the test substance becomes indirectly coupled to the microplate for testing purposes. Thus, the test substance may be “held down” by the interactive substance in some way. The test substance may interact with the interactive substance by binding specifically to the interactive substance. The interactive substance may be a first molecule and the test substance may be a second molecule. Thus, the system is operable to provide information concerning the second molecule in the sample due to the interaction between the first and second molecules on the test portion of the microplate. The interactive substance may be a first biomolecule and the test substance may be a second biomolecule. The interactive substance may be an antibody (or antibodies) and the test substance may be the associated antigen. In one embodiment, the interactive substance may comprise or be an aptamer and the test substance may comprise a target molecule associated with that aptamer. Aptamers are oglionucleic acid or peptide molecules that bind to specific target molecules, thus making them particularly suitable for the present system and method. In another embodiment, the interactive substance may comprise or be complementary DNA (cDNA).

In one embodiment, at least the test portion of the microplate is coated with a biocompatible coating to enhance adhesion of the interactive substance to the test portion.

Advantageously, the test portion comprises silicon, and the surface roughness of the test portion is less than 200 nm. More advantageously, the surface roughness of the test portion is within the range 3-20 nm.

In order to perform multiple tests at the same time, the microplate may further comprise one or more additional test portions. Each additional test portion includes a respective additional interactive substance that is inherently interactive with a respective additional test substance. The microplate is arranged such that at least the test portion and the one or more additional test portions of the microplate may be brought into contact with the sample. The processor is further operable to process the received sensory data time series so as to provide information about each additional test substance in the sample based on the sensed interaction between that additional test substance and the respective additional interactive substance on the respective additional test portion of the microplate.

Another way of performing multiple tests at the same time involves the microplate and the plurality of mutually spaced sensors together form a testing apparatus. The system then further comprises at least one further testing apparatus for providing information about at least one further test substance in the sample.

The system may further comprise a container for receiving the sample. The microplate is disposed relative to the container such that, when the sample is received in the container, at least the test portion of the microplate is submerged in the sample. The handheld testing device may further include the container. The container may be a micro-well or micro-channel in a substrate. The microplate is incorporated in the substrate such that, when the sample is dropped onto the micro-well or micro-channel, surface tension acts to draw the sample into the micro-well or micro-channel thereby submerging at least the test portion of the microplate in the sample.

The system may further comprise an aspirating device operable to remove the majority of the sample from the microplate, such that the remainder of the sample on the microplate substantially consists of any of the test substance which has interacted with the interactive substance on the test portion of the microplate.

According to a second aspect of the present invention, there is provided a method of testing a sample. The method comprises the steps of: providing a microplate having a test portion including an interactive substance, wherein the interactive substance is inherently interactive with a specified test substance; bringing the test portion of the microplate into contact with the sample; vibrating the microplate; providing a plurality of mutually spaced sensors coupled to the microplate; obtaining a respective sensory data time series from each sensor during vibration of the microplate, the microplate and the sensors being arranged such that the obtained sensory data time series are not independent from one another; and processing the sensory data time series so as to provide information about the test substance in the sample based on the sensed interaction between the test substance and the interactive substance on the test portion of the microplate.

The method may further comprise the step of outputting to a user the test results obtained in the processing step. The step of bringing the test portion of the microplate into contact with the sample may comprise submerging the test portion of the microplate in the sample.

The method may further comprise the step of removing the majority of the sample from the microplate, such that the remainder of the sample on the microplate substantially consists of any of the test substance which has interacted with the interactive substance on the test portion of the microplate. The step of removing the majority of the sample from the microplate may comprise aspirating the sample from the microplate. The steps of vibrating the microplate, obtaining sensory data time series, and processing the sensory data time series may be performed after the step of removing the majority of the sample from the microplate.

### BRIEF DESCRIPTION OF THE DRAWINGS

FIG. 1a is a schematic plan view of a prior art biosensing platform as described in WO2011/001138;

FIG. 1b is a schematic side view of the prior art biosensing platform of FIG. 1a;

FIG. 2 is a schematic perspective view of another prior art biosensing platform as described in WO2011/001138;

FIG. 3 is a Scanning Electron Microscope (SEM) image of a prior art integrated biosensing platform as described in WO2011/001138;

FIG. 4 is a schematic representation of a sample testing system in accordance with one embodiment of the present invention;

FIG. 5 is a schematic representation of a handheld testing device which forms part of the sample testing system of FIG. 4 in one embodiment of the present invention;

FIG. 6 is a schematic representation of a microplate including multiple test portions which may be used in the sample testing system of FIG. 4; and

FIG. 7 is a schematic representation of a multiple microplate system which may be used in the sample testing system of FIG. 4.

FIG. 8 is a schematic representation of various chips arrangements with different numbers of microplates and test portions which may be used in the sample testing system of FIG. 4.

### DETAILED DESCRIPTION OF A PREFERRED EMBODIMENT

WO2011/001138 in the name of Aston University relates to a method of and system for characterising a property or behaviour of at least one biological cell. The method and system may be used, for example, to characterise cell properties and behaviour such as cell propagation, cell polarity, cell movement, cell growth, cell contraction, cell migration, cell proliferation, cell differentiation, and microbe growth in vitro. The method described in WO2011/001138 comprises the steps of: providing a microplate; submerging at least one surface of the microplate in a cell culture medium such that at least one biological cell to be characterised is in contact with the microplate; vibrating the microplate; providing a plurality of mutually spaced sensors coupled to the microplate; obtaining a respective sensory data time series from each sensor during vibration of the microplate, the microplate and the sensors being arranged such that the obtained sensory data time series are not independent from one another; and processing the sensory data time series so as to characterise a property or behaviour of the at least one biological cell.

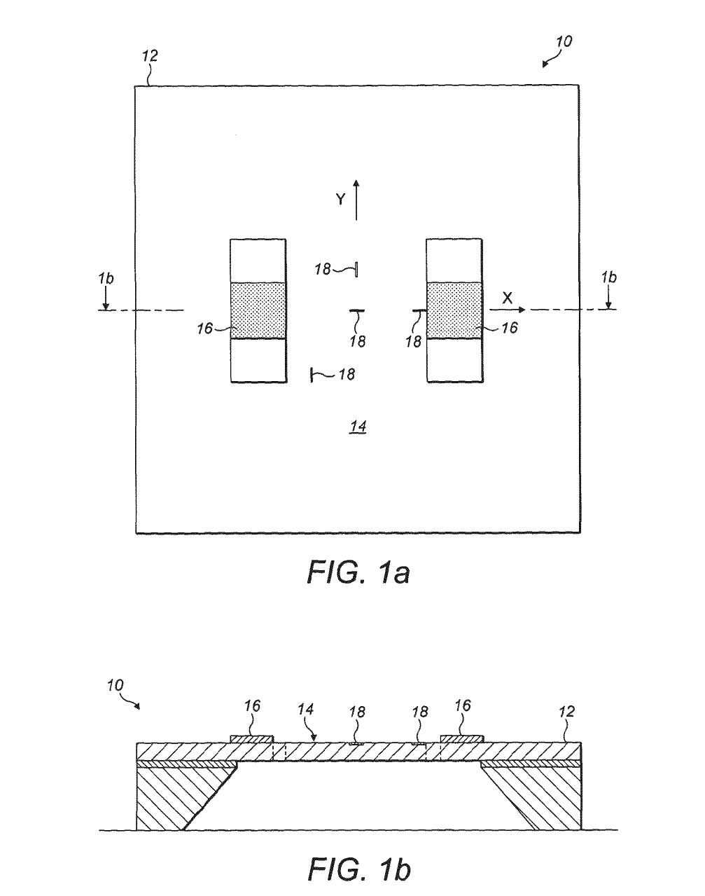

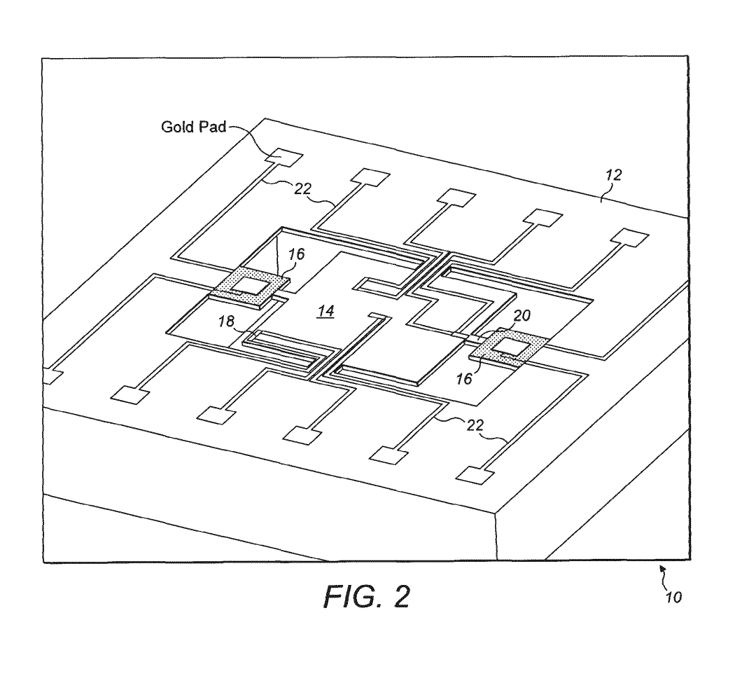

The micro/nano-scale biosensing system of WO2011/001138 includes a biosensing platform disposed within a container of cell culture fluid. FIGS. 1a and 1b show a plan view and a side view of one embodiment of a biosensing platform 10 described in WO2011/001138. A slightly different embodiment is shown in perspective view in FIG. 2.

The biosensing platform 10 of WO2011/001138 is largely formed from an SIO substrate 12. The biosensing platform 10 includes a microplate 14, two actuators in the form of piezoelectric transducers (PZTs) 16, four mutually spaced sensors 18, and a power input (not shown). The biosensing system of WO2011/001138 further includes a processor (not shown). The biosensing platform 10 is designed to be able to work in fluid (e.g. water), with a good bio-sensitivity under the high damping conditions. The biosensing platform 10 is either single-sidely or double-sidely immersed in cell culture fluid within the fluid container to maintain the natural cell living environment. The biosensing platform 12 uses materials that are biocompatible, such as silicon and gold, such that biological cells can use it as a natural growth ground when it is submerged in cell culture fluid.

The microplate 14 of WO2011/001138 is a thin micromachined membrane (plate/diaphragm) which acts as a micro/nano-scale sensing platform. The microplate 14 is deformable. The microplate 14 has dimensions in the range of tens to hundreds of microns in the X- and Y-directions. For example, the microplate 14 may have dimensions from tens to thousands of microns (e.g. 100-400 μm) in the X- and Y-directions. The depth of the plate in the Z-direction is about 3 μm as shown in FIG. 1b, but a depth in the range of a few nanometers up to tens of microns would also be appropriate. These dimensions are representative rather than limiting. The microplate 14 may be supported by means of a variety of different boundary conditions (e.g. clamped, cantilever, free and point supported, etc.). In the embodiment of FIG. 2 of WO2011/001138, the microplate 14 is rectangular. The microplate 14 is supported by four hinges 20, each hinge being located centrally along a respective one of the four sides of the microplate 14. This is one example of the microplate boundary conditions.

Actuators (i.e. excitation sources) are used to vibrate the microplate 14 of WO2011/001138 within the cell culture fluid. In the embodiment of FIGS. 1a and 1b, the actuators are two PZT (Lead Zirconate Titanate) thin films 16. The PZTs 16 are deposited inside or beside the region of the microplate 14 to provide powerful excitation force with limited energy consumption. Thus, the biosensing system is designed to be capable of self-excitation. As an alternative to the use of PZT actuators, the microplate 14 may instead be actuated by sound excitation. The actuators 16 may be integrated into the biosensing platform 10. The actuators 16 may be integrated into the microplate 14.

The biosensing system of WO2011/001138 is designed to be capable of self-sensing. Four distributive Piezoresistive-gauge (PZR) sensors 18 are shown in FIGS. 1a and 2. The sensors 18 are placed at well-selected locations for obtaining the whole-domain dynamical/vibrational information of the microplate 14. The sensors 18 may be embedded in the microplate 14. Advanced micro-fabrication techniques are used to produce the sensing elements 14 and associated connecting tracks 22 shown in FIG. 2. As an alternative to the use of PZR sensors, different sensor types may be used, e.g. optical, strain, or acceleration sensors. The sensors 18 may be integrated into the biosensing platform 10. The sensors 18 may be integrated into the microplate 14. The positions of the sensors 18 can be optimised with regard to maximising sensitivity for discrimination and maximising the range of high performance over the microplate surface.

The PZT actuators 16 and piezoresistive-gauge sensors 18 of WO2011/001138 are of good compatibility with CMOS circuits and are easily integrated with other electronic components. The electronic parts of the biosensing platform 10 of WO2011/001138 (e.g. the electrode wires, gold pads, and connecting probes) are sealed with biocompatible material. The whole biosensing platform 10 is packaged by using standard DIL (Dual in-line). The signal flow (input and output signals) may be processed either through external processing instruments or internal electronic chips.

Advanced tools and processes are used for the micro/nano fabrication of the biosensing platform of WO2011/001138, including optical and electron beam lithography, plasma etching and a focused ion beam tool capable of etch and deposition for rapid prototyping in nanofabrication.

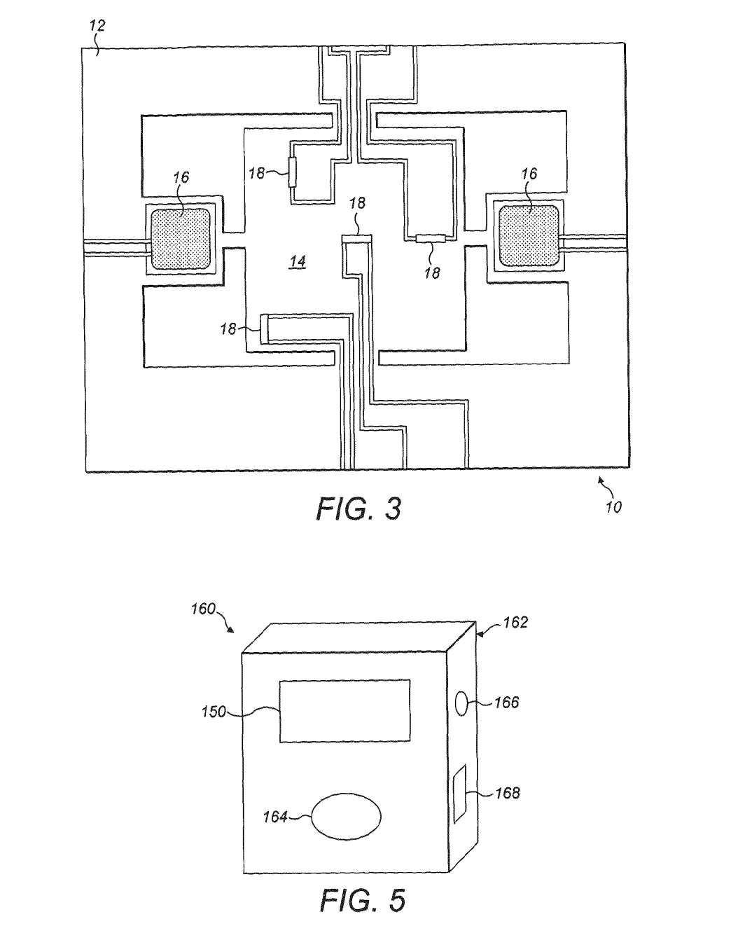

FIG. 3 is a Scanning Electron Microscope (SEM) image of an integrated microsystem (i.e. the biosensing platform 10) of WO2011/001138 based on a 100 μm square sensing membrane, which was manufactured with distributive piezoresistive sensors (i.e. the sensors 18) and PZT actuators (i.e. the actuators 16). Such a microsystem enables the device to be capable of self-sensing and self-excitation. This microsystem can be embedded into an electronic circuit to build a lab-on-chip system.

In use, the biosensing system of WO2011/001138 is used to discriminate a single cell or a collected group of cells' property or behaviour.

The cell culture fluid container of WO2011/001138 is partially or completely filled with cell culture fluid. The microplate 14 of the biosensing platform 10 of WO2011/001138 is placed into the cell culture fluid container such that at least one surface of the microplate 14 is submerged or immersed in the cell culture fluid. For example, the microplate 14 may be completely submerged within the cell culture fluid. Alternatively, only the bottom surface of the microplate 14 may be submerged within the cell culture fluid. The submersion of the microplate 14 within the cell culture fluid enables biological cells within the cell culture fluid to use the microplate 14 as a natural growth ground. Thus, there is in contact with the microplate 14 at least one biological cell whose property/behaviour is to be characterised by the biosensing system and method.

The microplate 14 of WO2011/001138 is then vibrated by the actuators (e.g. PZTs 16). The microplate 14 can be excited periodically (e.g. using a sinusoidal function) or randomly with a wide frequency band random signal (e.g. Pseudo Random Binary Signals, white noise, or burst random, etc.). The type of excitation/vibration will vary depending on the implementation purposes. The microplate 14 is vibrated because the contacting biological cells do not impose a significant force on the microplate 14 on their own. The contacting biological cells impact on the mass, stiffness and strain properties of the microplate 14. Thus, measurements of these variables (e.g. strain gauge measurements) may be used to quantify the effect of the contacting biological cells on the microplate 14 and to thereby infer the properties/behaviour of the cells to be characterised. Using a static microplate 14, the deflection of the microplate 14 by the contacting cells is very small which makes it difficult to detect the signals in, for example, the field of strain in the microplate 14. Consequently, it can be difficult to infer the properties/behaviour of the cells to be characterised. Thus, the microplate 14 is advantageously vibrated towards and away from the contacting biological cells so as to produce a stronger signal in the field of strain in the microplate 14 due to the presence of the cells. Alternatively/additionally, the microplate 14 may be vibrated in other directions rather than solely towards and away from the contacting biological cells. Vibrating the microplate 14 has other advantages too: the vibrations provide additional information about the dynamical character of the microplate 14 (e.g. natural frequency shifts, mode shape changes, and other nonlinear coupling effects). This additional dynamical information may also be used to characterise properties or behaviour of the contacting cells.

Whilst the microplate of WO2011/001138 is being vibrated, respective sensory data time series are obtained from each sensor 18. The microplate 14 is a continuous medium that provides a nonlinear coupling between the contacting biological cells and the sensors 18. Thus, the sensors 18 are coupled through the deformation response of the microplate 14 to biological cells in contact with the microplate 14 such that the sensory data time series are not independent from one another. This means that, although the sensors 18 receive local sensory data from the microplate 14, the sensory data time series from a particular sensor 18 may show cell movement remote from that sensor 18. In other words, the sensors 18 indirectly sense properties/behaviour of the biological cells via the microplate 14. The biosensing platform uses the variation of its dynamic/vibrational characteristics as the information source to sense the surface-contact biological cells and particles. By interpreting the simultaneous collective sensed responses of the sensors 18, the nature of any cell disturbance can be discriminated in such a way as to determine a property or behaviour of the contacting biological cells. The sensors 18 respond in a non-independent (i.e. coupled) manner due to the presence of the microplate 14, which acts as the coupling mechanism between the sensors 18. Due to the coupled nature of the system, only a relatively small number of discrete sensors 18 are needed on the microplate 14. The resolution of the biosensing platform 10 of WO2011/001138 is not limited to the pitch separating the sensors 18 and can therefore be used to detect variations much smaller than the smallest scale of manufacturing. Furthermore, due to the coupled nature of the system, the sensors 18 may be provided on a surface of the microplate 14 other than the cell-contacting surface. This adds to the robustness of the approach.

Having obtained the sensory data time series in WO2011/001138, these time series are processed using advanced system identification methodologies and embedded IT tools so as to characterise a property or behaviour of the at least one biological cell. During the processing step, the sensory data time series from each sensor is processed together with the sensory data time series from each of the other sensors (i.e. the data is processed collectively). The processing is nonlinear. For example, nonlinear signal processing techniques such as neural networks or Karhunen-Loeve decomposition may be used to process the coupled simultaneous time series, either in time or frequency domain.

The nonlinear processing model of WO2011/001138 utilises the dynamical information in the sensory data time series to detect cell properties and behaviour. Spatial dynamical information from the microplate 14 of WO2011/001138 (e.g. mode shapes, coupling between the sensors, etc.) is used to derive spatial dynamical information regarding the cells on the microplate 14 (e.g. polarity, stem cell growth). System identification tools are used to correlate the output of the processing step with the property or behaviour of the cell/cells/tissues which it is desired to characterise. In other words, the dynamical information will be correlated to the state and characteristics of the dynamical cell properties. For example, the property or behaviour of interest may be one that is essential in drug development, microbiological and tumour screening, or stem cell biology. This can include static or dynamic properties or behaviours, such as propagation, polarity, cell movement/growth, contraction, migration, proliferation or differentiation and microbe growth in vitro. The system and method of WO2011/001138 may be deployed to derive size, shape and movement information on the contact of a cell or cells during the processes of cell culture, cell manipulation and cell surgery. One aim of the biosensing method and system of WO2011/001138 is to detect the change in cell morphology, migration, proliferation, differentiation, and contractility during cell culture and growth processes. The dynamic characteristics of the microplate 14 (such as velocities and accelerations) are used to infer the information required using the relatively few sensing elements 18 through system identification algorithms. The dynamic response signals of the microplate 14 (i.e. the sensed data time series) are applied to intelligent time series identification algorithms to derive the desired cell property or behaviour information. Discrimination of the cell properties/behaviour is achieved by using embedded information tools. Outputs can be in discrete form or continuous with a variety of descriptors according to the aims of the application.

The nonlinear processing model used in the biosensing system of WO2011/001138 is trained using training data. The microplate 14 of WO2011/001138 vibrates differently under different loading conditions. Therefore, the nonlinear processing model takes into account the known dynamics of the microplate 14 in a liquid environment. For example, the microplate dynamics will be affected by the acoustic pressure waves caused by the interaction of the microplate 14 with the cell culture fluid (which generally has a slightly higher density than water). Thus, the nonlinear processing model is built with results from using a micro scanning laser vibrometer to investigate the micro scaling effects on the dynamics and sound radiation of the microplate 14 in fluid. The micro scanning laser vibrometer is used to measure the dynamics and sound radiation of the microplate 14 in liquid, such as natural frequencies, natural modes, forced response at certain forcing conditions. Thus, use of the micro scanning laser vibrometer enables an appropriate nonlinear processing model (e.g. neural network) to be created. In other words, results obtained using the micro scanning laser vibrometer are used as training data for the neural network, for example. Through the simulation of dynamics of a submerged microplate, the nonlinear processing model can be set up to infer the loading conditions from the sensory data time series of the vibrating microplate 14. In the modelling process, the displacements/velocities/accelerations at different sensing positions can be obtained through simulation. The nonlinear processing model that relates the parameters extracted from the dynamic signals (i.e. the sensory data time series) to the external forces/loadings is set up using system identification techniques such as Karhunen-Loeve decomposition, wavelet analysis and artificial neural network methods. The nonlinear processing model can be tested and validated by experiments using the Pseudo-Random Binary Sequence (PRBS) excitation and identification method. The advantage of PRBS signals is that they possess the property where their autocorrelation function is a close approximation to the impulse function. The dynamics of all frequencies are excited by PRBS signals. Thus dynamics of the microplate 14 under any forcing conditions can be derived. The validated model can then be used to deduce the force/loading applied on the microplate 14 by the sensory data time series of the vibration at different locations on the microplate 14. When the system identification technique is applied to cell/tissue monitoring, the state and condition of cell dynamics is deduced. The acceleration amplitude of the microplate 14 is less when the microplate 14 is submerged, this is due to the fact that each mode generates an acoustic pressure in the plane of the microplate 14, the normal modes become coupled in liquid. Still, by placing the sensors 18 in appropriate positions, the principal mode shapes can be related to the loading and dynamic conditions on the microplate 14 through proper system identification techniques. The correlation of cell behaviour to the transients detected and information derived from the microplate sensing surface is taken into account in the nonlinear processing model of WO2011/001138.

WO2011/001138 further describes applications and experimental results in connection with the described biosensing system. Fabrication of membrane biosensing devices is described. The dynamics information of each membrane biosensing device is measured in the forms of a series of frequency response functions (FRFs). Biological experiments and a neural network method are also described in detail. These details will not be repeated here, but many of the methodologies are equally applicable to the present invention.

The present invention modifies the biosensing platform 10 of WO2011/001138 for use in a sample testing system and method, as described further below. Unless otherwise stated, it is intended that the various embodiments, methodologies, and modifications described above with reference to WO2011/001138 are equally applicable to the present invention.

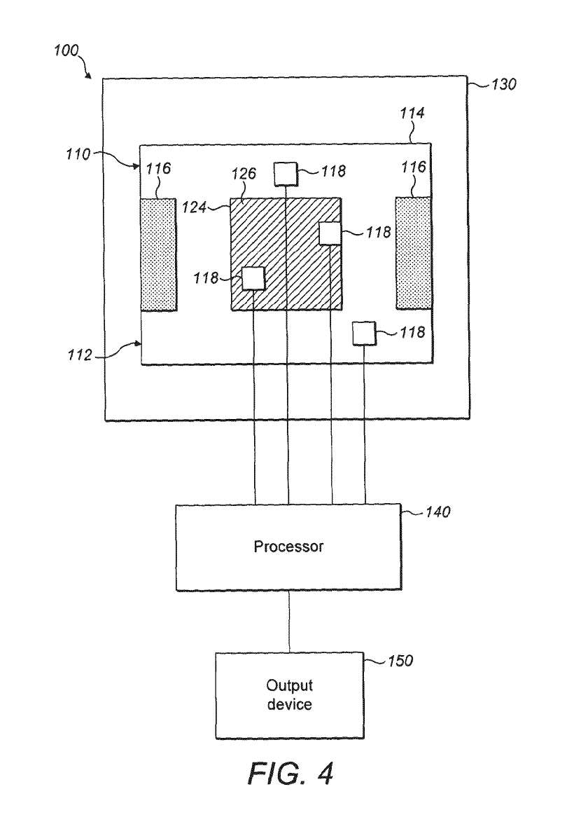

FIG. 4 shows a sample testing system 100 in accordance with one embodiment of the present invention. The sample to be tested is a fluid sample, such as a bodily fluid sample of urine, blood, saliva, semen, etc. The sample may be subject to pre-processing (e.g. filtration using a microfluidic filter) prior to testing using the sample testing system 100. Alternatively, the sample testing system 100 may be used to test the raw sample. The sample testing system 100 is used to test for a specific test substance, such as a specific antigen or other biomolecule, in the sample. In the specific embodiment described below, it will be assumed that the sample testing system 100 is being used to test for a specific antigen in a blood sample. However, other tests are clearly envisaged within the scope of the invention.

It should be noted that antigens do not exist in samples in isolation. Instead, they are found on the surface of other biomolecules (e.g. bacteria, protein, etc.). Thus, whilst we refer herein to the test substance being an antigen, it will be understood that the test substance actually includes the antigen and the biomolecule on whose surface the antigen is found. Thus, when testing for a specific antigen in a blood sample, we are in fact also testing the blood sample for the biomolecule (e.g. bacteria, protein, etc.) on which the specific antigen is found. The sample testing system 100 includes a sensing platform 110 in a container 130, a processor 140 and an output device 150. The container 130 is intended to receive the blood sample to be tested, thereby bringing the blood sample into contact with at last part of the sensing platform 110 in use. The sensing platform 110 shares many similarities to the biosensing platform 10 of WO2011/001138, namely that the sensing platform 110 is largely formed from a substrate 112 similar to the substrate 12, and that the sensing platform 110 includes a microplate 114 similar to the microplate 12, two actuators 116 similar to the PZTs 16, four mutually spaced sensors 118 similar to the sensors 16, and a power input (not shown). The substrate 112 may be a silicon-based substrate (e.g. an SIO substrate as in WO2011/001138). Silicon-based substrates are very common, so are readily available and well understood. In a potentially cheaper alternative embodiment, the substrate 112 may be a polymer substrate. Other substrates may also be used. As in WO2011/001138, the actuators 116 are operable to vibrate the microplate 114.

The main difference between the sensing platform 110 of the present invention and the biosensing platform 10 of WO2011/001138 is that the microplate 114 of the sensing platform 110 has a test portion 124 including an interactive substance 126. The interactive substance 126 will be discussed further below. The test portion 124 may comprise the whole microplate 114. Alternatively, the test portion 124 may comprise one entire surface of the microplate 114. However, in the embodiment of FIG. 4, the test portion 124 comprises part of a surface of the microplate 114. In particular, the test portion 124 is a central part of one side of the microplate 114.

Prior to adding the interactive substance 126 to the test portion 124 of the microplate 114, at least the test portion 124 may be coated using gold, silicon or a polymer, for example, so as to provide a biocompatible surface that is suitable for adherence by the interactive substance 126. Multiple biocompatible coatings are also possible. For example, it is possible to provide a polymer or gold layer on top of silicon. Other biocompatible coatings are also envisaged within the scope of the invention. For example, a bio-coating, such as Protein A may be used. Protein A is well known for its ability to bind immunoglobulins. Other immunoglobulin-binding bacterial proteins such as Protein G, Protein A/G and Protein L may alternatively be used. The purpose of adding a biocompatible coating is to provide a good surface for the interactive substance 126 to adhere to. The coating may be applied to the test portion 124 alone, or to a larger portion of the microplate 114 (e.g. the whole microplate 114).

In addition, the surface of the test portion 124 may be prepared to have a particular surface roughness to further enhance the adhesive properties of the test portion 124 for the interactive substance 126. Surface roughness (or simply “roughness”) is a measure of the texture of a surface. It is quantified by the vertical deviations of a real surface from its ideal form. If these deviations are large, the surface is rough; if they are small the surface is smooth. Roughness plays an important role in determining how a surface will interact with its environment. In an article entitled “Surface Modification of Bio-MEMS Micro-device with Conducting Polymer—Studies with Rat Cardiomyocytes” (Proceedings of the 2005 IEEE Engineering in Medicine and Biology 27th Annual Conference, Shanghai, China, Sep. 1-4, 2005), Lin et al. find that surface roughness can be affected by coatings, such as gold biocompatible coatings as mentioned above. In an article entitled “Influence of nanoscale surface roughness on neural cell attachment on silicon” (Nanomedicine: Nanotechnology, Biology, and Medicine 1, 2005, pp 125-129), Khan et al. find that surface roughness has considerable influence on neural cell attachment on bare silicon. They conclude that from an average roughness of 0 to 64 nm, cell adherence increases with roughness; but at roughness of around 204 nm or more, roughness negatively affects attachment. In an article entitled “Morphology and adhesion of biomolecules on silicon based surfaces” (Acta Biomaterialia 1, 2005, pp 327-341), Bhushan et al. provide patterning on a silica surface in order to absorb a streptavidin protein onto the surface. In particular, perpendicular lines at spacings of 400 nm with depths of 15-20 nm and widths near 45 nm were “drawn” in 25 μm×25 μm² on the 5 mm×5 mm samples. In an article entitled “Tuning cell adhesion by controlling the roughness and wettability of 3D micro/nano silicon structures” (Acta Biomaterialia 6, 2010, pp 2711-2720), Ranella et al. found that optimal cell adhesion was obtained for small roughness ratios, independently of the surface wettability and chemistry. The roughness ratio was calculated by dividing the actual, unfolded, surface area of spikes over the total irradiated area. Thus, it is clear that surface roughness impacts on the adhesive qualities of a surface. When the test portion 124 comprises silicon, the surface roughness of the test portion 124 is advantageously less than 200 nm. More advantageously, the surface roughness of the test portion 124 is within the range 3-20 nm. In one embodiment, the surface roughness of the test portion 124 is around 10 nm. A chemical mechanical polishing (CMP) method may be used to reduce the surface roughness of the test portion 124 if desired.

As in WO2011/001138, the sensors 118 are mutually spaced and are coupled to the microplate 114. Each sensor 118 is operable to provide a respective sensory data time series during vibration of the microplate 114. The microplate 114 and the sensors 118 are arranged such that the provided sensory data time series are not independent from one another. The sensors 118 may be coupled to the test portion 124 of the microplate 114. Alternatively, the sensors 118 may be coupled to another portion of the microplate 114. In the embodiment of FIG. 4, two of the four sensors 118 are disposed within the test portion 124 of the microplate 114 and two of the four sensors 118 are disposed outside the test portion 124 of the microplate 114.

The microplate is arranged such that at least the test portion 124 of the microplate may be brought into contact with the blood sample during testing. In the embodiment of FIG. 4, this arrangement is accomplished by disposing the microplate 114 within the container 130 such that, when the blood sample is received in the container 130, at least the test portion 124 of the microplate 114 is submerged in the blood sample. In an alternative embodiment (not shown in FIG. 4), the container 130 may comprise a micro-well or micro-channel in the microplate substrate 112 such that, when the sample is dropped onto the micro-well or micro-channel, surface tension acts to draw the sample into the micro-well or micro-channel thereby submerging at least the test portion of the microplate in the sample. In a further alternative embodiment, surface tension could be used to retain the sample on the microplate even in the absence of a container, micro-well or micro-channel.

As mentioned above, the test portion 124 of the microplate 114 includes an interactive substance 126. The interactive substance 126 is inherently interactive with a specified test substance, which is itself the subject of the test. In other words, the sample testing system 100 is used to test a fluid sample for the specified test substance. In the present example, the interactive substance 126 is an antibody coating of a type of antibodies which interact with the specific antigens/biomolecules that are being tested for in the blood sample. In this case, the “interaction” between the interactive substance 126 and the test substance is the bonding of the antibodies on the test portion 124 of the microplate 114 with antigens in the blood sample.

It is envisaged that other interactive substances could be used depending on the required test. For example, when testing for a first molecule in the sample, the interactive substance may be a second molecule which is known to inherently interact with the first molecule. In this case, the system is operable to test for the presence/quantity of the first molecule in the sample due to the interaction between the first and second molecules on the test portion of the microplate. One type of molecules which may be used in the test is biomolecules. Antigens and antibodies are specific examples of biomolecules.

The interactive substance 126 is coupled to the test portion 124 of the microplate 114. In a preferred embodiment, the interactive substance 126 is coated onto the test portion 124 of the microplate 114 by means of a microfluidic device. The interactive substance may be directly or indirectly coated onto the test portion 124 of the microplate 114. In other words, there may be an intermediate layer (such as another coating) between the interactive substance 126 and the test portion 124. However, any intermediate layer should still allow coupling between the test portion 124 of the microplate 114 and the interactive substance 126. The interactive substance 126 (here the antibody coating) is effectively part of the microplate 114. It is possible to test for the specific antigen (and its associated biomolecule on which it is found) in the blood sample due to its interaction on the microplate 114 with the antibody coating 126. Instead of coating the interactive substance 126 onto the test portion 124 of the microplate 114, it may instead be bonded onto the test portion 124 by other chemical/biological methods. Other types of binding interaction used in the present system 100 may include (a) enzyme binding with substrate/inhibitor and (b) receptor binding with ligand/agonist/antagonist. Other non-binding interactions could be cell/microbe number and would depend on the inherent ability of cells to bind to non-coated surfaces—this has been demonstrated.

The processor 140 is operable to receive the sensory data time series from the sensors 118 and to process the received sensory data time series so as to provide information about the antigens/biomolecules in the blood sample based on the sensed interaction between the antigens and the antibody coating 126 on the test portion 124 of the microplate 114.

The output device 150 is operable to output to a user any test results received from the processor 140. The output device may include a display, a speaker, and/or a printer, for example.

As mentioned above, the sample testing system 100 is used to test for a specific test substance (e.g. antigens/biomolecules) in a fluid sample (e.g. a blood sample).

The container 130 is partially or completely filled with the blood sample such that the test portion 124 of the microplate 114 is in contact with the blood sample. For example, the microplate 114 may be completely submerged within the blood sample. Alternatively, only the test portion 124 surface of the microplate 114 may be submerged within the blood sample. The submersion of the microplate 114 within the blood sample enables antigens within the blood sample to interact with the antibody coating 126 on the test portion of the microplate 114. Antigens in the sample are brought into contact with the test portion 124 of the microplate 114 such that any antigens specifically associated with the type of antibodies coated onto the test portion 124 of the microplate 114 will “interact” with (i.e. bind to) the antibody coating 126. Thus, the specific antigens/biomolecules being tested for become coupled to the test portion 124 of the microplate 114.

As in WO2011/001138, the microplate 114 is vibrated using the actuators 116. Various types of actuators and various types of vibration are contemplated, as in WO2011/001138. The microplate 114 is vibrated because the contacting antigens/biomolecules do not impose a significant force on the microplate 14 on their own. The antigens/biomolecules bound to the microplate 114 by the antibodies 126 on the test portion 124 have an impact on the mass, stiffness and strain properties of the microplate 114. Thus, measurements of these variables (e.g. strain gauge measurements) may be used to quantify the effect of the antigens/biomolecules on the microplate 114 dynamics and to thereby infer properties and/or behaviour of the antigens being tested for.

Whilst the microplate 114 is being vibrated, respective sensory data time series are obtained from each sensor 118. As in WO2011/001138, the microplate 114 is a continuous medium that provides a nonlinear coupling between the antigens in the sample and the sensors 118. Thus, the sensors 118 are coupled through the deformation response of the microplate 114 to the antigens bonded to the microplate 114 such that the sensory data time series are not independent from one another. This means that, although the sensors 118 receive local sensory data from the microplate 114, the sensory data time series from a particular sensor 118 may show antigen signals remote from that sensor 118. In other words, the sensors 118 indirectly sense properties/behaviour of the antigens via the microplate 114. The sensing platform 110 uses the variation of its dynamic/vibrational characteristics as the information source to sense the surface-contact antigens. By interpreting the simultaneous collective sensed responses of the sensors 118, it is possible to identify, quantify, and characterize the antigens under testing. The sensors 118 respond in a non-independent (i.e. coupled) manner due to the presence of the microplate 114, which acts as the coupling mechanism between the sensors 118. Due to the coupled nature of the system, only a relatively small number of discrete sensors 118 are needed on the microplate 114. The resolution of the sensing platform 110 is not limited to the pitch separating the sensors 118 and can therefore be used to detect variations much smaller than the smallest scale of manufacturing. Furthermore, due to the coupled nature of the system, the sensors 118 may be provided on a surface of the microplate 114 other than the test portion 124 which binds the antigens. This adds to the robustness of the approach.

Having obtained the sensory data time series, these time series are processed in the processor 140 using advanced system identification methodologies and embedded IT tools so as to provide information regarding antigens bound to the antibodies coated onto the test portion 124 of the microplate 114. During the processing step, the sensory data time series from each sensor 118 is processed together with the sensory data time series from each of the other sensors 118 (i.e. the data is processed collectively). The processing is nonlinear. For example, nonlinear signal processing techniques such as neural networks or Karhunen-Loeve decomposition may be used to process the coupled simultaneous time series, either in time or frequency domain.

The nonlinear processing model described in WO2011/001138 may be used to derive spatial dynamical information regarding the antigens bound to the microplate 114. For brevity, details of the nonlinear processing model of WO2011/001138 will not be repeated here. Nonetheless, it will be understood that the “contacting cells” being analysed in WO2011/001138 are equivalent to the bound antigens in the specific embodiment of the present invention which has been described above. The nonlinear processing model may be trained using training data as in the biosensing system of WO2011/001138. Again, details will not be repeated here.

The processing by the processor 140 preferably occurs with little or no user interaction. In other words, the processing is preferably fully automated.

Outputs via the output device 150 can be in discrete form or continuous with a variety of descriptors according to the aims of the test. For example, it may be desirable to simply output a positive or negative test result regarding whether any of the specific antigens have been identified in the sample based on the processing of the sensory data time series. In other words, the test result is simply an indication of the presence (or not) of the specific antigen in the sample. Alternatively/additionally, it may be desirable to provide information regarding the level/amount/quantity/behaviour of those antigens in the blood sample. Alternatively/additionally, information can be output as to whether or not the quantity of the specific antigens in the blood sample exceeds a particular threshold. Again, a positive or negative result may be output in this case.

In one embodiment, we envisage a disposable microplate 114/sensing platform 110 for use in analysing a single blood sample. In this case, the disposable microplate 114/sensing platform 110 would be replaced with another disposable microplate 114/sensing platform 110 after a single use. Alternatively, the microplate 114/sensing platform 110 may be re-usable for analysing a plurality of blood samples. In this case, the microplate 114/sensing platform 110 would be able to be cleaned between uses. It may be cleaned in situ (acting as a permanent fixture) or it may be cleaned during removal of the microplate 114/sensing platform 110 from the system 100. It may be cleaned with an appropriate cleansing wipe.

FIG. 5 shows a handheld testing device 160 in accordance with an embodiment of the present invention. The handheld testing device 160 has a housing 162 which may be ruggedized for use in the field. The handheld testing device 160 includes a section 164 for receiving a sample (e.g. a blood sample). The sample-receiving section 164 includes the sensing platform 110 (not shown). The sample-receiving section 164 may comprise the container 130 (not shown). Alternatively, the container may be omitted and a very small sample (e.g. of blood) is instead received onto a surface of the microplate 114 by means of a pin prick or the like, and is then held there by surface tension. The handheld testing device 160 of FIG. 5 further includes a display screen 150. The display screen may indicate when the handheld testing device 160 is switched on, for example.

The handheld testing device 160 may further include the processor 140 as an internal processor (not shown). In this case, the screen 150 may act as the output device 150 by outputting test results to a user from the processor 140.

Alternatively, the processor 140 may be separate from the handheld testing device 160. For example, the processor 140 could be the processor of a desk top computer, or a lap-top computer, or a smart phone. In this case, the output device 150 may also be separate from the handheld testing device 160. In one “separate processor” embodiment, the handheld testing device 160 is arranged to be coupled to the processor 140 by means of a coupling 166. The coupling 166 may be wired (e.g. by USB cable, Local Area Network, or using another connector cable/wire) or may be wireless (e.g. a Bluetooth connection, IEEE 802.11 or Wi-Fi connection). In another “separate processor” embodiment, the handheld testing device 160 is arranged to write the sensory data time series on a removable memory component (not shown). The removable memory component may then be removed from the handheld testing device 160 and read by the separate processor 140. In this embodiment, the handheld testing device 160 includes a suitable input 168 to receive the removable memory component. Similarly, the processor 140 also includes a suitable input to receive the removable memory component. The removable memory component may be a memory stick, a memory card, a CD, a DVD, an external hard drive, a USB memory stick, or an SD card, for example.

In the embodiment described above, the test portion 124 of the microplate 114 is immersed in the fluid sample during vibration of the microplate and during acquisition of the sensory data time series. Thus, the acquired sensory data time series related to data acquired whilst the test portion 124 of the microplate 114 is in solution. However, in an alternative embodiment, it is envisaged that the majority of the sample be removed from the microplate 114 in order to measure the residual bound test substance. In one such case, the system 100 further comprises an aspirating device (not shown) operable to remove the majority of the sample from the microplate, such that the remainder of the sample on the microplate substantially consists of any of the test substance which has interacted with the interactive substance on the test portion of the microplate. In the above described antigen-antibody test, residual bound antigens would remain on the test portion 124 of the microplate 114 with the majority of the blood sample being aspirated. The dynamical analysis would then be performed on the microplate 114 after aspiration. In other words, the steps of vibrating the microplate, obtaining sensory data time series, and processing the sensory data time series would be performed after the step of removing the majority of the sample from the microplate. Nonetheless, this does not preclude earlier vibration of the microplate 114 whilst it is still in the blood sample, since this may help to bring antigens in the blood sample into contact with the antibody coating 126 on the test portion 124 of the microplate 114.

In the embodiment described above, a single test is performed for a specific test substance (i.e. a specific antigen) using only a single microplate 114 with a single test portion 124 having a single interactive substance 126 (i.e. the antibodies corresponding to the antigens being tested for). However, the system 100 of the present invention may easily be extended to perform multiple tests.

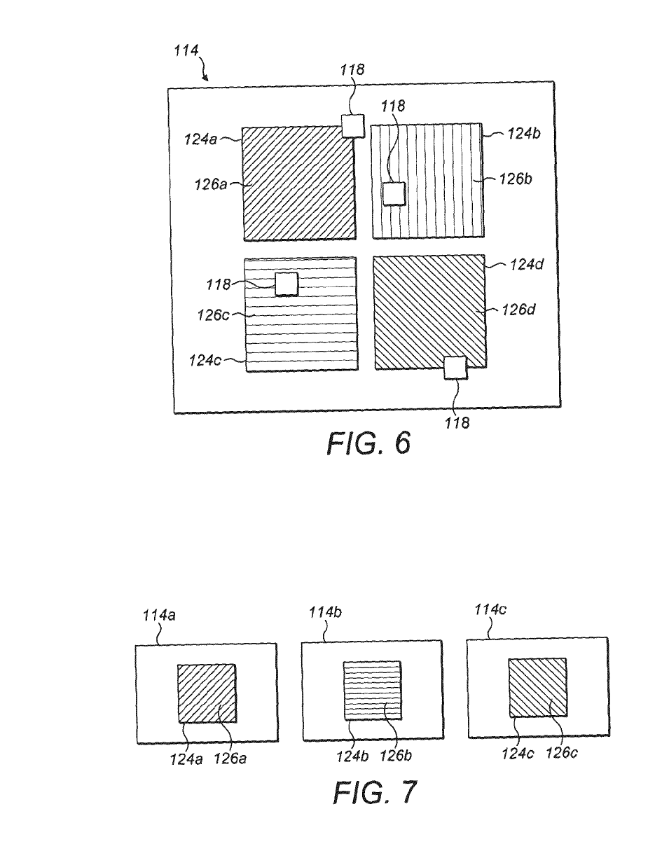

A first multiple-test embodiment is shown in FIG. 6. In this case, there is still a single microplate 114 with four sensors 118. However, the microplate 114 includes four test portions 124a-d. Each test portion 124a-d in FIG. 6 includes a respective interactive substance 126a-d that is inherently interactive with a respective test substance. The four test portions 124 are mutually spaced (i.e. remote from one another) such that they occupy separate, discrete, non-overlapping portions of the microplate 114. It would be possible to have the test portions 124 disposed directly adjacent one another, but a separation between the test portions is preferable. In FIG. 6, all of the test portions are disposed on a single side of the microplate 114, but one or more could be disposed on the other side if desired. In any case, the microplate 114 is arranged such that at least the test portions 124 may be brought into contact with the sample. Four test portions 124 are shown in FIG. 6, but any number of test portions 124 could be used so long as it is possible to deposit the relevant interactive substances 126 onto the test portions 124 with sufficient accuracy. It should be noted that technology suitable for depositing the interactive substances 126 onto the test portions 124 is readily available.

The microplate 114 of FIG. 6 may be used in the system 100 so as to test for up to four different test substances. Since the microplate 114 acts as a nonlinear coupling between the sensors 118 and the bound test substances, and since the sensors 118 are coupled through the deformation response of the microplate 114 to the bound test substances such that the sensory data time series are not independent from one another, the processor 140 is able to process the acquired sensory data time series so as to differentiate between the test substances bound to the four separate test portions 124 of the microplate 114. Thus, the four tests may be performed simultaneously. Physical separation (i.e. spacing) between the test portions is preferred since this aids the distinction between the different test portions in the processing step. Thus, the processor 140 is operable to process the received sensory data time series so as to provide information about each test substance in the sample based on the sensed interaction between that test substance and the respective interactive substance 126 on the respective test portion 124 of the microplate 114.

A second multiple-test embodiment is shown in FIG. 7. In this case, there are three separate microplates 114a-c, each with its own set of sensors 118 (not shown) and its own test portion 124a-c. Each test portion 124a-c has a respective interactive substance 126a-c that is inherently interactive with a respective test substance. The three microplates 114 are mutually spaced in the same plane. However, the microplates 114 could instead be stacked or arranged in any other configuration relative to one another. The three microplates 114 may be disposed in the same container (or micro-well or micro-channel), or may alternatively each be disposed in a separate container (or micro-well or micro-channel). In any case, the three microplates 114 are arranged such that at least the test portions 124 may be brought into contact with the sample. Each microplate 114 may have its own actuator(s) 116 (not shown) for vibration purposes. Alternatively, the same actuator(s) 116 may be used to vibrate all three microplates 114. Three microplates 114 are shown in FIG. 7, but any number of microplates 114 could be used in a single system 100. The microplate 114 of FIG. 7 may be used in the system 100 so as to test for up to three different test substances simultaneously.

In a third multiple-test embodiment (not shown), the embodiments of FIGS. 6 and 7 may be combined so as to provide multiple microplates 114 each including multiple test portions 124. Again, this increases the number of test which may be performed simultaneously.

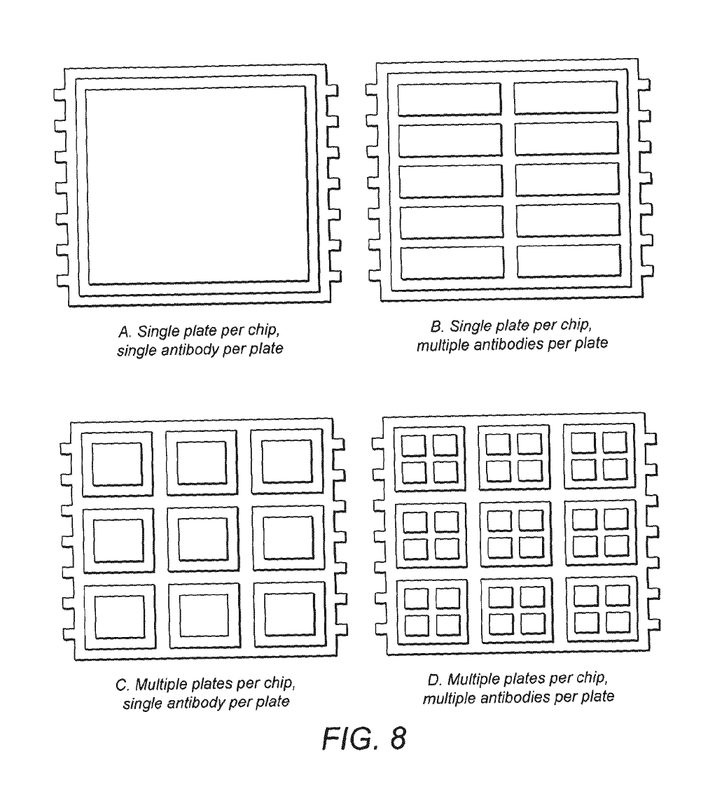

FIG. 8 provides further examples of various chips with different test portion/microplate arrangements within the scope of the present invention. FIG. 8A shows a single microplate per chip with a single test portion per microplate. FIG. 8B shows a single microplate per chip with multiple test portions per microplate (this is somewhat similar to the arrangement of FIG. 6). FIG. 8C shows multiple microplates per chip with a single test portion per microplate (this is somewhat similar to the arrangement of FIG. 7). FIG. 8D shows multiple microplates per chip with multiple test portions per microplate.

Thus, the present invention provides, amongst other things, a handheld testing device 160 which may be used for point of care testing in GP clinics. The device 160 contains a BioMEMS microplate biosensing component (namely the sensing platform 110). This microplate 114 of the sensing platform 110 is coated with antibodies (i.e. the interactive substance 126) that interact with corresponding antigen-based biomolecules. If it does not have its own internal processor, the device 160 may be connected (via Bluetooth or USB) to a computer, which runs diagnostic software. The software uses data gathered by the microplate 114 to identify, quantify, and characterize the biomolecules, enabling a rapid and cost effective diagnosis. Various models of device 160 could be made available, each offering a family of related tests, e.g. pregnancy, STD, common infectious diseases (e.g. flu), cancer, etc. dependent on the interactive substance(s) coated on the microplate(s) 114.

The present invention provides advantages over current GP clinic testing methodologies in terms of being easier to use, cheaper, quicker, and more cost effective. For example, it is envisaged that a handheld testing device may be manufactured for only £10-15, with single-use disposable sensing platforms eventually costing only a few pence each. Thus, costs to GP clinics should be reduced by an order or magnitude for each test.

It will be understood that the systems and methods of the present invention could be used by a GP in distinguishing between a viral infection and a bacterial infection in a patient. All that would be needed would be a microplate 114 including the relevant antigen(s) for the virus and/or bacteria in question. Thus, by using such a test, a GP would have much greater certainty as to whether or not to prescribe antibiotics (which are only effective against bacterial infections).

Rather than implementing as a handheld device 160, a bench-scale testing system is also envisaged within the scope of the invention.

One specific example of an antibody-antigen interaction which may be used in a test according to the present invention is described below.

The antigen in question is CEA (carcinoembryonic antigen). Literature shows T84.1 and T84.66 should be a good antibody to bind orthogonal epitopes on CEA. From the literatures, sensing surface Si or SiO2 can be functionalised: (1) CEA antibodies could be functionalised on the surface using silica sol-gel glass, with uniform pore size antibody can be bound to properly with CEA in the pores, or (2) attachment of a lipid bilayer on the SiO2 surface, the lipid layer is biotinylated, biotin-avidin chemistry can be used to conjugate a biotinylated capture of antibody on the surface, or (3) another promising method is to derivatise the surface using triethoxysilane, for covalent bonding of antibodies.

Whilst the systems and methods for testing a sample have been described with reference to medical testing of bodily fluid sample, it will be understood that the present systems and methods may also be readily used for testing in other fields. For example, other potential fields of use include water testing, food testing, agricultural/veterinary testing, defence, etc.

Although preferred embodiments of the invention have been described, it is to be understood that these are by way of example only and that various modifications may be contemplated.

1. A system for testing a sample, the system comprising:
a microplate having a test portion including an interactive substance, wherein the interactive substance is inherently interactive with a specified test substance, the microplate being arranged such that at least the test portion of the microplate may be brought into contact with the sample;
at least one actuator operable to vibrate the microplate;
a plurality of mutually spaced sensors coupled to the microplate, each sensor being operable to provide a respective sensory data time series during vibration of the microplate, the microplate and the sensors being arranged such that the provided sensory data time series are not independent from one another; and
a processor operable to receive the sensory data time series from the sensors and to process the received sensory data time series so as to provide information about the test substance in the sample based on the sensed interaction between the test substance and the interactive substance on the test portion of the microplate.

2. The system of claim 1 wherein the test portion of the microplate is coated with the interactive substance.

3.-11. (canceled)

12. The system of claim 1 wherein the test substance interacts with the interactive substance by binding specifically to the interactive substance.

13. The system of claim 1 wherein the interactive substance is a first molecule and the test substance is a second molecule.

14. The system of claim 13 wherein the interactive substance is a first biomolecule and the test substance is a second biomolecule.

15. The system of claim 14 wherein the interactive substance is an antibody and the test substance is a respective antigen-biomolecule pair, the antigen being found on the surface of the biomolecule.

16. The system of claim 1 wherein the interactive substance comprises an aptamer and the test substance comprises a target molecule associated with that aptamer.

17. The system of claim 1 wherein the interactive substance comprises cDNA.

18. The system of claim 1 wherein the microplate is a silicon microplate or a polymer microplate.

19. The system of claim 1, wherein at least the test portion of the microplate is coated with a biocompatible coating to enhance adhesion of the interactive substance to the test portion.

20. The system of claim 1, wherein the test portion comprises silicon, and the surface roughness of the test portion is less than 200 nm.

21. The system of claim 1, wherein the surface roughness of the test portion is within the range 3-20 nm.

22. The system of claim 1 wherein:
the microplate further comprises one or more additional test portions, each additional test portion including a respective additional interactive substance, wherein each additional interactive substance is inherently interactive with a respective additional test substance, the microplate being arranged such that at least the test portion and the one or more additional test portions of the microplate may be brought into contact with the sample, and
the processor is further operable to process the received sensory data time series so as to provide information about each additional test substance in the sample based on the sensed interaction between that additional test substance and the respective additional interactive substance on the respective additional test portion of the microplate.

23. The system of claim 1 wherein the microplate and the plurality of mutually spaced sensors together form a testing apparatus, and wherein the system further comprises at least one further testing apparatus for providing information about at least one further test substance in the sample.

24. The system of claim 1 further comprising a container for receiving the sample, the microplate being disposed relative to the container such that, when the sample is received in the container, at least the test portion of the microplate is submerged in the sample.

25. (canceled)

26. The system of claim 15 wherein the container is a micro-well or micro-channel in a substrate, the microplate being incorporated in the substrate such that, when the sample is dropped onto the micro-well or micro-channel, surface tension acts to draw the sample into the micro-well or micro-channel thereby submerging at least the test portion of the microplate in the sample.

27. (canceled)

28. A method of testing a sample, the method comprising the steps of:
providing a microplate having a test portion including an interactive substance, wherein the interactive substance is inherently interactive with a specified test substance;
bringing the test portion of the microplate into contact with the sample; vibrating the microplate;
providing a plurality of mutually spaced sensors coupled to the microplate; obtaining a respective sensory data time series from each sensor during vibration of the microplate, the microplate and the sensors being arranged such that the obtained sensory data time series are not independent from one another; and
processing the sensory data time series so as to provide information about the test substance in the sample based on the sensed interaction between the test substance and the interactive substance on the test portion of the microplate.

29. (canceled)

30. The method of claim 28 wherein the step of bringing the test portion of the microplate into contact with the sample comprises submerging the test portion of the microplate in the sample.

31. The method of claim 28 further comprising the step of removing the majority of the sample from the microplate, such that the remainder of the sample on the microplate substantially consists of any of the test substance which has interacted with the interactive substance on the test portion of the microplate.

32. (canceled)

33. The method of claim 31 wherein the steps of vibrating the microplate, obtaining sensory data time series, and processing the sensory data time series are performed after the step of removing the majority of the sample from the microplate.
